(19) United States
(12) Patent Application Publication
(10) Pub. No.: US 2020/0361984 A1
(43) Pub. Date: Nov. 19, 2020

(54) CLICK BASED LIGATION

(71) Applicant: BASECLICK GMBH, Neuried (DE)

(72) Inventors: Thomas FRISCHMUTH, Berlin (DE); Sascha SERDJUKOW, Kiefersfelden (DE)

(21) Appl. No.: 16/651,450

(22) PCT Filed: Sep. 28, 2018
(86) PCT No.: PCT/EP2018/076495

§ 371 Date: Mar. 27, 2020

(30) Foreign Application Priority Data
Sep. 29, 2017 (EP) 17194093.5
Mar. 26, 2018 (EP) 18164071.5

(51) Int. Cl. C07H 21/02; C07H 21/04; C12Q 1/6806; C12Q 1/6869

(52) U.S. Cl. CPC C07H 21/02; C07H 21/04; C12Q 2523/109; C12Q 1/6869; C12Q 1/6806

(57) ABSTRACT

The present invention relates to new methods and reagents for coupling molecules by a so-called click reaction in the presence of a suitable catalyst and a metal cation. Further, the invention relates to an activator composition for such click ligation reaction, a click ligation reagent kit, a device for performing such click ligation reaction, and the use of such method, composition, reagent kit and device to improve the efficiency of coupling of molecules via a click reaction, especially in the context of next generation nucleic acid sequencing methods.

The present invention relates to new methods and reagents for coupling molecules by a so-called click reaction in the presence of a suitable catalyst. Further, the invention relates to an activator composition for such click ligation reaction, a click ligation reagent kit, a device for performing such click ligation reaction, and the use of such method, composition, reagent kit and device to improve the efficiency of coupling of molecules via a click reaction, especially in the context of next generation nucleic acid sequencing methods.

### BACKGROUND OF THE INVENTION

In 2001/2002 the groups of Sharpless and Meldal independently defined the concept of “Click chemistry” and the criteria for a transformation to be considered as a “Click” reaction (Sharpless, K. B. et al, Angew. Chem. 2002, 114, 2708; Angew. Chem. Int. Ed. 2002, 41, 2596, Meldal, M. et al, J. Org. Chem., 2002, 67, 3057). Since then, the copper catalyzed reaction of azides with alkynes to give 1,2,3-triazoles, a variation of the 1,3-dipolar Huisgen cycloaddition (R. Huisgen, 1,3-Dipolar Cycloaddition Chemistry (Ed.: A. Padwa), Wiley, New York, 1984) has become the most widely used Click reaction. As a result of its mild conditions and high efficiency, this reaction has found a myriad of applications in biology and material sciences, such as e. g. DNA labeling purposes (Gramlich, P. M. A. et al., Postsynthetic DNA Modification through the Copper-Catalyzed Azide-Alkyne Cycloaddition Reaction. Angew. Chem. Int. Ed. 2008, 47, 8350).

The rapid analysis of genetic material for a specific target sequences, e.g. for the presence of single nucleotide polymorphism, the presence of a certain gene like a resistance gene, or of mRNA requires easy to use, efficient and reliable tools. A major problem is the need to detect DNA or RNA of interest directly in small biological samples such as patient blood or plants. These provide the analyte only in minute amounts. In order to reach the required sensitivity, an amplification step is usually required wherein either the nucleic acid analyte is amplified prior to analysis or a detection method is used in which the minute detection signal, directly obtained from the DNA/RNA analyte, is amplified.

Methods for the amplification of the nucleic acid analyte include PCR and other nucleic acid amplification protocols. PCR amplification has the major advantage that, within a pool of different DNA strands obtained from a biological material, only the DNA sequence of interest is amplified. This is the basis for a reliable analysis of single genes in complex biological samples.

Enzymatic ligation of oligonucleotides is a standard procedure in numerous protocols for oligonucleotide manipulation and is required for sequencing, cloning and many other DNA- and RNA-based technologies. The enzymes involved in the catalysis of the ligation reaction form phosphodiester bonds between 5′-phosphate ends of DNA or RNA and 3′-hydroxyl ends. In order to work efficiently, the ligases require double-stranded oligonucleotides, which are preferably pre-organized by using cohesive ends or even splint strands. The ligation reaction can join any 5′-phosphate with any 3′-hydroxyl end, since the reaction is not sequence specific. This lack of substrate specificity is a major advantage for a broad general application of enzymatic ligations and has contributed to its wide application. Ironically, this is at the same time an immense challenge for many sequencing applications as sequence artifacts are generated from unspecific joining of oligonucleotide fragments. As almost every sequencing technology requires ligation of a known oligonucleotide sequence, the so-called adapters, for primer binding during sequencing and PCR amplification, this can generate artifactual recombination, sequence chimera formation (DNA-RNA strands for RNA sequencing) and adapter dimers (a pair of ligated adapters with no insert sequence) formation.

In order to reduce sequence artifacts, the 3′-end of the primer is blocked by insertion of a 2′,3′ dideoxynucleotide (ddNTP). This avoids adapter dimers formation and ensures correct directional ligation, but it cannot influence artifactual recombination and chimera formation. These effects need to be corrected using special algorithms during contig (a set of overlapping sequence data reads) generation from sequence reads.

The present invention is concerned with a click chemistry-based method for the non-enzymatic joining of molecules, especially oligonucleotides, which is efficient, site-specific and is compatible with PCR amplification. The reaction between two non-templated single-stranded oligonucleotides is more efficient than the comparable enzymatic ligation reaction, thus e.g. eliminating the need for second strand synthesis in e.g. RNA sequencing.

The preparation of alkyne- or azide-containing oligonucleotides has been studied extensively. Of special interest for the present invention are “backbone mimics”, i.e. non-natural alternatives for the phosphodiester bond, which can be generated by copper-catalyzed azide alkyne cycloadditions (CuAAC). Some of the resulting triazole-containing oligonucleotides can be converted into natural phosphodiester backbones by polymerase enzymes without mutation of the sequence and thus are fully biocompatible (Shivalingam et al., Molecular Requirements of High-Fidelity Replication-Competent DNA Backbones for Orthogonal Chemical Ligation (2017) doi:10.1021/jacs.6b11530). FIG. 1 shows the structure of some triazole backbone mimics that are generated by click chemistry, the natural phosphodiester is shown on the left side for comparison.

An especially useful application of this artificial DNA backbone technology lies in sample preparation for next-generation sequencing. Routh et al. (ClickSeq: Fragmentation-Free Next-Generation Sequencing via Click Ligation of Adaptors to Stochastically Terminated 3′-Azido cDNAs. J. Mol. Biol. 427, 2610-2616 (2015)) provide a protocol for RNA sequencing, in which an adapter oligonucleotide is clicked to a 3′-azide terminated cDNA fragment and a PCR reaction is performed to provide a cDNA library. In comparison with a standard library preparation protocol that involves enzymatic ligation of the adapter oligonucleotide, this method can drastically decrease the rate of artifactual recombination and sequence chimera formation.

A severe limitation to this application of the click technology in DNA or RNA sequencing so far is a less than satisfactory efficiency in which the 3′-azido-blocked cDNA fragments used in the method are captured during library generation. Only about 10% of the 3′-azido-blocked cDNA fragments could be ligated to an alkyne-modified oligonucleotide. Furthermore, the conversion of this triazole-linked single-stranded DNA into a double stranded DNA via read-through of the triazole linkage is not efficient. While for standard RNAseq experiments, such efficiency might be sufficient, for other applications requiring a thorough capture of the original RNA or DNA, the efficiency of the click reaction and the subsequent read-through is considered inadequate and must be improved.

Accordingly, it is an object of the present invention to provide means to achieve a higher efficiency especially of the click ligation in order to establish successful application of the click technology to the next generation sequencing.

### SUMMARY OF THE INVENTION

The present invention relates to improving the efficiency of ligating two reaction partners in a click reaction using an alkyne group and an azide group as the click functional groups. The click reaction as hereinafter intended is a reaction between a 1,3-dipolar moiety, which is an azide, with an unsaturated moiety, which is a terminal alkyne, catalyzed by a metal catalyst, especially a heterogeneous Cu(I) or another suitable metal catalyst. The click reaction in the catalyzed form is a non-concerted ionic mechanism which results in a 5-membered heterocyclic 1,2,3-triazole moiety.

According to a first aspect, the present invention relates to a method for coupling a first molecule to a second molecule in a click reaction, wherein the first molecule comprises a first click functional group which is an alkyne group, and the second molecule comprises a second click functional group, which is an azide group, the method comprising contacting the first and second molecules in a reaction mixture in the presence of a catalyst, preferably a heterogeneous Cu catalyst, the method being characterized in that the click reaction is performed in the presence of additional metal cations in the reaction mixture.

A further aspect of the present invention is an activator composition for use in a click reaction, wherein a first molecule comprising a first click functional group, which is an alkyne group, and a second molecule comprising a second click functional group, which is an azide group, are coupled in the presence of a catalyst, preferably a heterogeneous Cu catalyst, said activator mixture comprising metal cations, Cu(I)-stabilizing ligands and optionally an organic solvent.

Still a further aspect of the present invention is a click ligation reagent kit comprising at least as one component a catalyst and as a second component an activator composition according to the present invention.

Still a further aspect of the present invention relates to a device having at least one reaction chamber, wherein the reaction chamber comprises a catalyst, preferably a heterogeneous Cu catalyst for carrying out a click reaction between two reaction partners and further wherein metal cations or an activator composition of the present invention are contained in the reaction chamber.

Still a further aspect of the present invention relates to the use of the above methods, compositions, reagent kits and devices for efficiently ligating molecules, preferably oligonucleotides via a click reaction. An example of such molecules carries a detectable label. The ligation can thus provide labeled biomolecules which may be used for detecting an analyte, e.g. a nucleic acid in a sample, particularly involving the use of a compound labeled by a click reaction, which forms an association product with the analyte to be detected. In particular, the methods, compositions, reagent kits and devices can be used in next generation sequencing technologies, including RNA library preparation, DNA library preparation, and in the analysis of complex oligonucleotide mixtures, especially parallel and multiplex RT-qPCR applications. Further, the methods, compositions, reagent kits and devices of the present invention can be used for sequencing by ligation methods (e.g. “Solid Sequencing” from ABI), in ligase chain reactions, in cloning methods, in particular to form a recombinant DNA, and in gene synthesis.

The research that led to the present invention has shown that surprisingly addition of metal ions to a click reaction which is catalyzed by a catalyst, in particular by a Cu(I) catalyst, especially a heterogeneous Cu (I) catalyst, can vastly improve especially the oligonucleotide-oligonucleotide click reaction kinetics. This improvement allows efficient click reactions between the backbone functionalized oligonucleotides in yields that were until now only obtained by preorganization of the reaction partners through splint oligonucleotides.

### DETAILED DESCRIPTION OF PREFERRED EMBODIMENTS

The present invention provides for improved conditions for click reactions, which avoid the need for splint oligonucleotides and still lead to high yields of the click ligation reaction. In an example of such click reaction which can be performed under the conditions of the present invention, a 3′-alkyne- or a 3′-azide-modified nucleotide, can be introduced into an oligonucleotide, e.g. by enzymatic incorporation during reverse transcription (RNA sequencing) or during blunting (single stranded DNA overhangs are filled with nucleotides and/or removed) and/or by dA tailing (non-templated addition of nucleotides, most often dATP, to the 3′ end of blunt double-stranded DNA). For RNA sequencing, a first adapter (adapters are short oligonucleotides containing complementary sequences for primer binding and hybridization) can be introduced using a partly randomized primer. A second adapter is click ligated to the 3′-alkyne or 3′-azide terminated cDNA. A schematic presentation of such reaction during ClickAdapt RNA library preparation is shown in FIG. 2a.

Especially in the context of such RNA sequencing, it is possible and can even be preferable to employ adapters containing complementary sequences for primer binding and hybridization which contain at their 5′ end a sequence which is complementary to a more downstream region of the adapter. Inverted palindromic sequences or inverted repeats are examples for such complementary sequences. Hybridization of these complementary parts of the adapter sequence leads to formation of a double-stranded loop at the 5′ end of the adapter. Using such an adapter containing a double-stranded loop at the 5′ end ensures that the adapter does not hybridize with other sequences on either the same or another RNA contained in the reaction mixture. A corresponding reaction is schematically presented in FIG. 2b).

While in addition to the double-stranded part, a single-stranded loop area is present in such adapters, such loop area is not likely to cause unspecific hybridization. Nevertheless, it is preferred to keep such loop area small. Preferably the loop area comprises less than 10 nucleotides, more preferably less than 6 nucleotides and most preferably the loop area comprises only 3 nucleotides.

For DNA sequencing the first adapter is click ligated to the enzymatically 3′-alkyne or 3′-azide modified oligonucleotide (the second adapter is introduced during PCR via a partly complementary binding region (e. g. 12 mer) of the adapters). The respective ClickAdapt DNA library preparation workflow is shown in FIG. 3. The primer or the adapter can contain an index sequence to allow for a subsequent correlation to one of different samples. This allows for applying the click sequencing technology to mixtures of several enzymatically produced samples. After removal of excess modified nucleotide, a click ligation is performed between the enzymatically produced and purified modified oligonucleotides and a 5′-alkyne- or 5′-azide modified oligonucleotide acting as an adapter in order to introduce nucleotide sequences that are necessary for PCR amplification and/or sequencing. To this end, the adapter oligonucleotide preferably is produced via solid phase synthesis and the 5′-modification introduced at the end of such synthesis.

As the alkyne modification element, commercially available building blocks can be used, e.g. having the following structure:

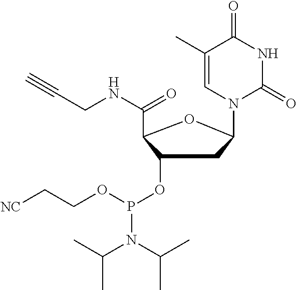 [SMILES: C#CCNC(=O)[C@H]1O[C@@H](n2cc(C)c(=O)[nH]c2=O)C[C@H]1OP(OCCC#N)N(C(C)C)C(C)C]

As a catalyst for the click reaction, a metal catalyst, preferably a Cu catalyst, and most preferably a heterogeneous Cu(I)-catalyst is used. In particular, a catalyst as described in EP 2 416 878 B1 can serve as the source for the catalytic active copper species for the click reaction. It is especially referred to paragraphs [0029] to [0031] of EP 2 416 878 B1, in which a detailed description of such catalysts is provided.

In the present invention, the click reaction comprises a copper catalyzed variation of a formal (3+2) cycloaddition between the azide and the terminal alkyne group. The irreversible formation of 1,2,3-triazoles as a result of the azide/alkyne reaction is biorthogonal due to the lack of azides and terminal alkynes in organisms, the required chemical groups are small (incorporation with minimal disruption of the biomolecule's environment) and the reaction is regioselective, resulting in exclusive formation of the 1,4 regioisomer.

The following reaction is the basis of the click ligation of azides and terminal alkynes

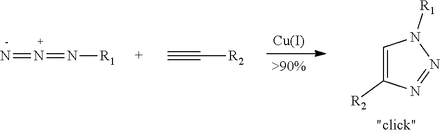

wherein R₁ and R₂ are first and second partners in the click reaction.

Click reaction conditions are known to the skilled person from e.g. the above cited prior art documents. Usually, the click reaction is performed in an aqueous reaction mixture at room temperature or slightly elevated temperature, preferably between 20 and 60° C., more preferably at 30-50° C., most preferably at 40-45° C. Depending on the temperature, the reaction will usually require an incubation time of from 10 minutes to several hours, preferably 30 minutes to 3 hours, and most preferably, 40-90 minutes. The reaction conditions are not critical and can be adjusted to the amounts and volumes of reagents used for the preparation of click products.

The catalyst is preferably a heterogeneous Cu-catalyst, more preferably a heterogeneous Cu(I)-catalyst. It should be noted, however, that other metal catalysts and especially other heterogeneous metal catalysts, such as Zr, W, Fe, Ru, Co, Th, Ir; Ni, Pd, Pt, Ag, Au, Zn, Cd, Hg and other metal ions have been reported to contribute directly or indirectly to a catalysis of the click reaction ligation and can also be used within the context of the present invention. Alternatively, also a homogenous Cu(I)-catalyst can be used for the click ligation. The heterogeneous Cu catalyst is elemental copper or a metal-C-catalyst, i.e. a solid Cu catalyst comprising a carbon-based support such as charcoal having incorporated Cu ions therein or elemental copper, which can generate Cu(I) ions. In an especially preferred embodiment, the heterogeneous Cu catalyst is a Cu(I)-C-catalyst which may be prepared as described by H. Lipshutz and B. R. Taft in Angew. Chem. Int. Ed., 2006, 45, 8235-8238.

The heterogeneous catalyst may be a particulate catalyst, e.g. a heterogeneous catalyst consisting of particles having a size of from 10 nm to 1000 μm, preferably from 100 μm to 800 μm. Alternatively, the catalyst may also be a porous non-particulate catalyst, e.g. a solid matrix having embedded therein catalytically active particles.

In a further embodiment, a further solid carrier material is included which is a material different from the heterogeneous Cu catalyst, e.g. a chromatographic material on which a biomolecule such as preferably a nucleic acid or a nucleic acid analog can be immobilized. Preferably, materials are included which allow for a separation and purification of the click reaction product from the other constituents of the reaction mixture. Exemplary mechanisms for separation and purification are size exclusion or affinity chromatography.

Examples of suitable chromatographic materials are an ion exchange material, a hydrophilic material or a hydrophobic material. In a preferred embodiment a hydrophilic material, e.g. silica gel, can be used in combination with the heterogeneous catalyst. In another preferred embodiment a hydrophobic material, e.g. silica C18 or C4 or an ion exchange resin, can be used in combination with the heterogeneous catalyst. In a still further preferred embodiment, the solid carrier material may be a resin which is used for the solid phase synthesis of biomolecules, especially nucleic acids and nucleic acid analogs.

It was found that the click reaction between an immobilized reaction partner and a reaction partner which is freely present in solution may be effectively catalyzed by a heterogeneous Cu catalyst system. This strategy may allow achieving simultaneously the click reaction and the purification and/or the separation of the product from the impurities and/or from excess reagents or salts eventually present in the reaction mixture.

In the method according to the present invention, the reaction mixture contains additional metal cations. Within the context of the present invention, the term “additional metal cations” refers to metal cations which are different from the catalyst metal present or emerging in cationic form during the click reaction.

Preferred metal cations are alkaline and earth alkaline metals, or other divalent metal ions with similar properties like e.g. zinc (Zn²⁺). In a preferred embodiment of the invention, such metal cations and especially such alkaline metals to be added to the reaction mixture are different from Na⁺. In an especially preferred embodiment of the present invention, divalent earth alkaline metal ions and in particular Mg²⁺ ions are present in the reaction mixture, which have been proven to be especially suitable cations for the anionic DNA phosphate backbone. Further preferred additional cations used for the purposes of the invention are Li⁺, K⁺, and Zn²⁺.

For this purpose, corresponding metal salts and especially Mg²⁺ salts are included in the reaction mixture. During the research leading to the present invention, it was surprisingly found that the addition of metal cations and especially Mg²⁺ to the reaction mixture causes all possible ligand positions for copper species on the phosphate backbone of the oligonucleotides to be occupied by the additional cations. As a consequence, the amount of copper species which is available as a catalyst in the reaction mixture is increased and the reaction kinetics is remarkably improved.

Suitable amounts of additional metal cations in the click reaction mixture are 1 to 200 mmol/l, preferably 5 to 25 mmol/l, especially preferably 10 to 20 mmol/l.

In the click reaction according to the present invention, it is preferred to further include a catalyst metal stabilizing ligand, especially a Cu and preferably a Cu(I) stabilizing ligand. As such ligand, known metal ligands, like amines and polytriazoles (T. R. Chan, R. Hilgraf, K. B. Sharpless and V. V. Fokin, Organic Letters, 2004, 6, 2853-2855.), especially Tris(3-hydroxypropyltriazolylmethyl)amine (THPTA), 2-(4-((bis((1-(tert-butyl)-1H-1,2,3,-triazol-4-yl)methyl)amino)methyl)-1H-1,2,3-triazolyl-1-yl)acetic acid (BTTAA), 2-(4-((bis((1-(tert-butyl)-1H-1,2,3,-triazol-4-yl)methyl)amino)methyl)-1H-1,2,3-triazolyl-1-yl) ethyl sulfate (BITES) (D. Soriano Del Amo, W. Wang, H. Jiang, C. Besanceney, A. C. Yan, M. Levy, Y. Liu, F. L. Marlow, P. Wu, J. Am. Chem. Soc. 2010, 132, 16893-16899.), Tris((1-benzyl-4-triazolyl)methyl)amine (TBTA) or analogs thereof having similar metal stabilizing properties can be used in the reaction mixture. These metal ligands stabilize e.g. the Cu(I) catalyst and protect the oligonucleotides against formation of reactive oxygen species. Further, addition of such metal ligands also improves the reaction kinetics of the click reaction.

Metal ligands are included in the click reaction mixture in an amount of 10 to 4000 μmol/l, preferably 500 to 1000 μmol/l and especially preferably 700 to 900 μmol/l.

In a further preferred embodiment of the present invention, an organic solvent is added to the reaction mixture. In particular, dimethyl sulfoxide (DMSO) can be advantageously included in the reaction mixture. Addition of such organic solvent and especially DMSO has a further positive effect on the efficiency of the click reaction between two oligonucleotides. It is assumed that DMSO disturbs the secondary structures of oligonucleotides and thus improves the accessibility of the functional groups of the reaction partners, e.g. the azide and alkyne groups. Within the context of the present invention, addition to a final content of 1 to 10% (v/v) of such organic solvent, especially DMSO, to the reaction mixture is considered useful, addition of organic solvents to a final content of 2 to 8% (v/v), and especially 4 to 6% (v/v) in the reaction mixture is preferred.

Surprisingly it was observed that the presence of additional metal ions, especially the presence of corresponding divalent metal ions in the reaction mixture of the click reaction causes a remarkable increase in click reaction product formation. Compared with the reaction performed without addition of such divalent metal cations, the yield of click product could be at least doubled or even tripled. Using the reaction mixture which further contains the organic solvent, especially DMSO, and a Cu-stabilizing ligand further improves the performance and leads to highly satisfactory results and yields of click product. These effects of the method according to the present invention carry forward to subsequent reactions and uses of such click products, especially to PCR reactions for DNA or RNA library preparation and next generation sequencing.

Considering the relevance of additional metal ions, especially divalent metal ions and some other substances for the efficiency of the click ligation, a further subject matter of the present invention is an activator composition for use in the click reaction for coupling molecules that are functionalized by on the one hand a terminal alkyne group and on the other hand an azide group in a Cu-catalyzed reaction, preferably a reaction carried out in the presence of a heterogeneous Cu catalyst. Such activator composition contains metal cations. As mentioned above, addition of such additional metal cations to the click reaction mixture, preferably earth alkaline metal cations and especially preferably Mg²⁺ ions, prevents binding of the copper catalyst species to the DNA or RNA phosphate backbone. Such binding occurs without addition of such metal cations and reduces the amount of catalyst available for the click reaction. Due to the presence of metal cations, especially divalent metal cations the blocking of the binding sites on the phosphate backbone, the amount of copper that is available as a catalyst is increased and the reaction kinetics improved.

As also already explained above in detail, the presence of Cu(I)-stabilizing ligands and/or the presence of an organic solvent, especially DMSO further improves the efficiency of the click reaction between the two oligonucleotides. Accordingly, a preferred activator composition according to the present invention contains the divalent metal cations and an organic solvent and at least one Cu-stabilizing ligand. Especially preferred is an activator composition containing as the divalent metal cation Mg²⁺ and DMSO and at least one Cu-stabilizing ligand selected from THPTA, BTTAA, analogs thereof or any mixtures of such ligands.

The activator composition contains these effector molecules in amounts which provide for a concentration in the click reaction mixture of the divalent metal cations of 1 to 200 mmol/l, preferably 5 to 25 mmol/l and especially preferably 10 to 20 mmol/l; the Cu-stabilizing ligand of 10 to 4000 μmol/l, preferably 500 to 1000 μmol/l and especially preferably 700 to 900 μmol/l; and/or the organic solvent, preferably DMSO of 1 to 10% (v/v), preferably 2 to 8% (v/v) and especially preferably 4 to 6%(v/v).

The activator composition according to the present invention can be provided as a composition containing one, two or all of the above-mentioned substances that help to improve the efficiency of the click reaction. The activator composition can be an aqueous composition containing a pre-dilution of the effector substances. The activator composition can also contain other solvents, especially further organic solvents or a combination of water and organic solvents. The activator composition can further contain other substances like buffer substances or any other substance that can be included in the performance of the click reaction.

A further subject matter of the present invention is a click ligation reagent kit, such reagent kit comprising at least as one component a catalyst as defined above, especially a Cu catalyst and preferably a heterogeneous Cu(I) catalyst, and as a second component an activator composition according to the present invention. As disclosed above in more detail, the activator composition has a distinct positive effect on the copper catalyzed click reaction in ligating alkyne-and azide-functionalized oligonucleotides. Accordingly, a reagent kit of the present invention provides the catalyst as well as the effector molecule(s) of the activator composition together in order to facilitate the implementation of the click ligation method disclosed herein. In preferred embodiments, the click ligation reagent kit contains as the activator composition the correspondingly preferred embodiment, wherein not only metal cations, preferably earth alkaline metal cations and especially Mg²⁺, but also at least one of an organic solvent and a Cu-stabilizing ligand is present. For best results, an activator composition containing all three effector substances is included in the click ligation reagent kit of the present invention.

The Cu(I) catalyst that is included in the inventive click ligation reagent kit preferably is a heterogeneous catalyst as described in EP 2 416 878 B1.

Optionally, such reagent kit contains further substances that are required components and/or advantageous in performing a click reaction. In addition to the above-mentioned components, a click ligation reagent kit of the present invention can further contain at least one azide- or alkyne-functionalized oligonucleotide, which can act as an adapter or primer. The reagent kit can contain labels, labeled click functional groups, modified and non-modified nucleotides, enzymes, buffers, adapters and/or primers, further carrier materials or chromatographic materials for purification, preferably materials as described above in the context of the click ligation method according to the invention and the various applications thereof. As mentioned above, depending on the reaction to be performed, adapters and primers can be included which contain complementary sequences which form a loop and a double-stranded area at the 5′ end of the adapter or primer.

In a further preferred embodiment of the present invention, the click ligation reagent kit can additionally contain substances and components required for a subsequent PCR reaction to amplify the click ligated product, for DNA or RNA library preparation and sequencing, e.g. for analyzing complex oligonucleotide mixtures.

A still further subject matter of the present invention is a device which facilitates performing the click reaction of the present invention. Such device comprises at least one reaction chamber, such reaction chamber comprising a catalyst, preferably a heterogeneous Cu catalyst for carrying out the click reaction between two reaction partners. Respective devices are disclosed and further described in EP 2 416 878 B1 and such disclosure applies also to devices which can be used according to the present invention.

According to the present invention, in addition to the presence of one or more components of a click ligation reaction, the device further comprises additional metal cations as defined above, especially divalent metal cations, preferably earth alkaline metal cations and especially Mg²⁺, or an activator composition as described above within the one or more reaction chambers of such device. Since the click reaction may be performed in very small volumes, the devices can be e.g. microtiter plate wells, pipette tips or spin columns, comprising one or more compartments which can act as the at least one reaction chamber. Metal ions or other preferred components employed according to the present invention are present in such devices in the at least one of the reaction chambers where the click reaction is performed.

It is to be understood that within the context of the present invention all different manners of performing such click reaction and all substances that are described for such purposes are included as long as the essential requirements of the methods, compositions, reagent kits and devices of the invention as described above are met. Accordingly, all other forms, variations and improvements of click reactions which are not explicitly described herein are considered applicable also within the context of the present invention as long as the click reaction mixture for the catalyzed click ligation includes the additional metal ions and especially the divalent metal cations which have been shown by the present invention to have a considerable advantageous effect on the efficiency and yield of the click reaction. While the presence of these cations, especially earth alkaline cations and most preferably Mg²⁺ ions is mandatory for solving the object of the present invention, other aspects of the click reaction can vary dependent on e.g. the nature of the molecules to be coupled by click ligation, the actually used catalyst, the reaction conditions and the intended uses and respective subsequent reactions to be performed.

The use of the methods, compositions, reagent kits and devices for efficiently ligating molecules, preferably oligonucleotides, via click reaction in order to provide click reaction products which can be used in subsequent reactions is a further subject matter of the present invention. As such subsequent reactions, all reactions are included that require ligation products which have a high purity and are provided in a sufficient lead high yield to perform such subsequent reaction. Examples of such subsequent reactions are PCR or other amplification methods for analyzing DNA or RNA samples, especially where complex oligonucleotide mixtures are to be analyzed. The present invention allows to obtain click ligation products of oligonucleotides constituting “backbone mimics”, i.e. of RNA or DNA molecules containing non-natural alternatives for phosphodiester bonds in their backbone. As it was found that such substances are fully biocompatible, the amplification of such molecules is highly effective and useful especially for the so-called next generation sequencing applications.

When the above stated conditions are applied e.g. to RNA sequencing library preparation, the second adapter can be efficiently click ligated to single stranded cDNA, thus making second strand synthesis needless and the total preparation time is reduced. Due to the bio-orthogonality and specificity of the click reaction, the click ligation occurs exclusively between azide- and alkyne-functionalized molecules. The azide is introduced stochastically during cDNA synthesis as the natural deoxynucleotides are supplemented with low concentration of e.g. 3′-azido-2 ′,3′-dideoxynucleotides, which terminates the fragments. The 5′-alkyne modified adapter oligo can be prepared by solid-phase DNA synthesis and can be used without purification, since the 5′-alkyne is incorporated last in solid phase synthesis and shorter non-labeled strands cannot be click ligated.

The inventive click concept can especially preferably be applied to DNA library preparation protocols. The DNA fragments are generally modified by blunting and dA tailing through DNA polymerase to allow more efficient subsequent enzymatic ligation. Replacing the natural dNTPs with e.g. 3′-azido-2′,3′-dideoxynucleotides during blunting or dA tailing introduces azide groups for click ligation to 5′-alkyne adapter oligonucleotides.

Application of the present invention in the context of the ClickSeq technology as described for example by Routh et al., vide supra, is also extremely valuable if complex oligonucleotide mixtures are to be analyzed. Parallel analyses for RNA-virus RNA can be done by performing multiplex RT-qPCR. For this purpose, several virus RNA specific primers (e.g. HIV, hepatitis C, etc.) are added to RNA in a sample for a reverse transcription reaction (e.g. a ClickAdapt reaction). Azide-terminated cDNA fragments are click ligated to an adapter to provide primer binding sites for subsequent PCR amplification. Due to the specificity of the click ligation, no artifactual recombination can occur, rendering separate qPCR setups for each detection unnecessary. Thus, the technology provides a basis for massive parallel RT-qPCR applications.

Application areas of this technology include all other DNA and RNA library preparation kits for sequencing, sequencing by ligation methods (e.g. “Solid Sequencing” from ABI), ligase chain reaction, cloning methods (to perform recombinant DNA) and gene synthesis, wherein two or more molecules are ligated via click functional groups. While partly in general and partly in more detail, possible applications have been described above, it is to be understood that other reactions including a click ligation performed under the conditions described herein are also included within the scope of the present invention.

### DESCRIPTION OF THE FIGURES

FIG. 1 shows the structure of some triazole backbone mimics generated by click chemistry (B-D) compared to the natural phosphodiester (A). Subsequent experimental examples in the context of PCR amplification are shown for the triazole backbone mimic (B) only;

FIG. 2a) shows a schematic ClickAdapt RNA library preparation which is one of the methods of interest in the present invention. It involves a combined cDNA synthesis, fragmentation and adapter click ligation. Second strand synthesis is obsolete since ssDNA can be efficiently click ligated;

FIG. 2b) shows the same ClickAdapt RNA library preparation as FIG. 2a) for which, however, a 1^{st} adapter comprising a double-stranded loop at the 5′ end is included.

FIG. 3 shows a schematic ClickAdapt DNA library preparation workflow which is a further method of interest in the present invention. A sample double-stranded (ds) DNA is fragmented and the fragments are manipulated by blunting and dA tailing like in a standard DNA library preparation. By using e.g. 3′-azido-2′,3′-dideoxynucleotides instead of natural dNTPs, 3′azide terminated dsDNA is obtained. After removal of excess nucleotides by fragment purification (including size selection), the first adapter is clicked to the fragment via its 5′-alkyne group. The second adapter is introduced during PCR amplification via a short (about 12 bp) 3′-sequence which is the reverse complement of the 5′-end of the first adapter and acts as the first primer for amplification;

FIG. 4 shows the influence of various cations on the click reaction efficiency and yield;

FIG. 5 shows PCR products of cDNA produced from a 3′-azide terminated cDNA and a 5′-alkyne adapter using different DNA polymerases; and FIG. 6 shows the result of Sanger sequencing of the amplified click ligated product of FIG. 5;

FIG. 7 shows exemplary structures of azide and alkyne modified nucleotides for enzymatic incorporation and subsequent use in click ligation;

FIG. 8 shows structures of exemplary 5′-ends of adapter oligonucleotides for click ligation (A-C). 5′-alkyne modified oligonucleotide (with the base B=thymine of structure A) was used for the examples 1, 2 and 4 (in FIGS. 4, 5 and 9). Structure B was used in the subsequent examples 1 and 4 (in FIGS. 4 and 9).

FIG. 9 shows the oligo-oligo click reaction yield for a low oligo concentration.

FIG. 10 shows an ethidium bromide stained agarose gel (3% in TAE) of PCR samples with template from click library preparation of eGFP mRNA. The template cDNA was generated using different nucleotide mixtures (dNTP constant 500 μM, various AzddNTP) during reverse transcription (rt). M=low molecular weight DNA marker (NEB), 1=dNTP only, 2=100 μM AzddNTP, 3=50 μM AzddNTP, 4=25 μM AzddNTP, 5=10 μM AzddNTP.

FIG. 11 shows an analytical HPL chromatogram of an oligo-dye CuAAC reaction using MgSO₄ as an additive.

FIG. 12 shows an analytical HPLC result from an oligo-oligo click crude reaction mix at 260 nm detection. The peak at 5.6 min corresponds to the alkyne modified oligo, the peak at 7.6 min corresponds to the azide modified oligo. The two peaks that are new after the click reaction (6.1 and 6.4 min) have the correct mass of the click product. About 80% of the integrated peaks had the ESI-MS confirmed mass of the click product.

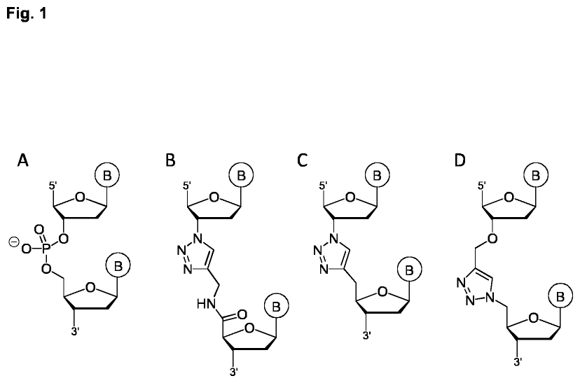

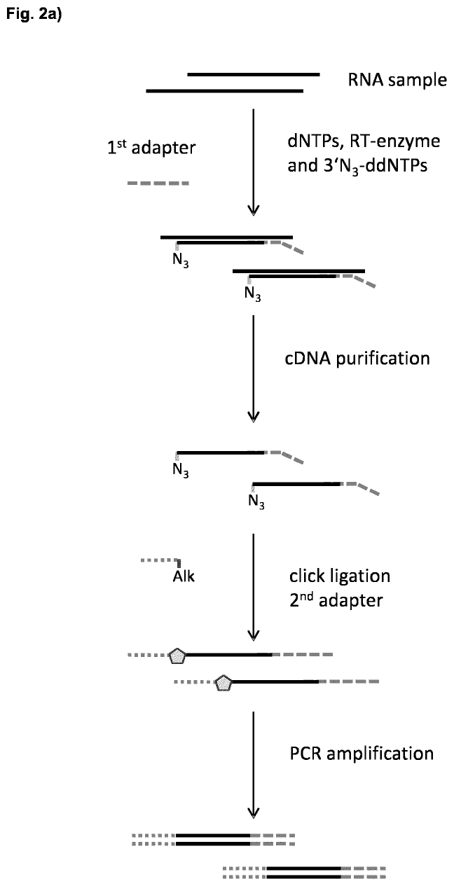

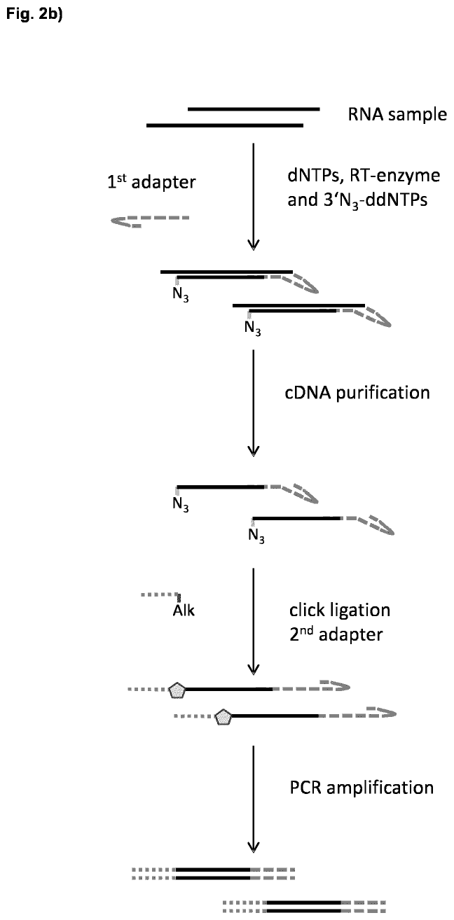

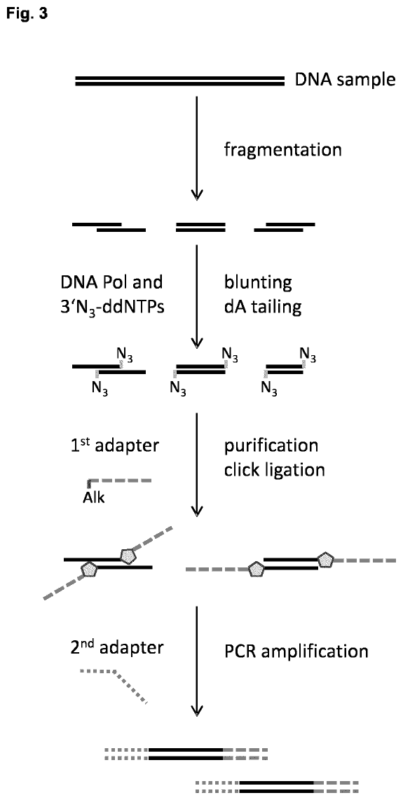

The following examples are provided for illustration purposes.

### EXAMPLE 1

### Preparation of Click Products

In a 200 μL reaction vial a single reactor pellet (600-800 μm, containing elemental copper) was combined with 12.5 μL reaction mix and incubated at 45° C. for 60 min. The reaction mix consisted of 4 mM THPTA, 55 μM of an alkyne oligol, 55 μM of an azide oligo1 and, when cation influence was studied 16 mM monovalent cations (or 8 mM divalent cation). dH₂O was used to adjust the volume to a final 12.5 μL if necessary.

After the incubation the sample was briefly spinned down and the supernatant was transferred to a new vial to stop the reaction. Samples were analyzed on 2.5% agarose gels (10×15 cm) prepared in TAE buffer (20 mM TRIS, 10 mM acetic acid, 0.5 mM EDTA).

Samples were prepared with 20% purple loading dye (NEB, New England BioLabs Inc.), and low molecular weight DNA ladder (25-766 bp, NEB, N3233) was prepared accordingly; usually 0.5 μL marker were used in 5 μL loading volume. Gels were run in TAE buffer applying constant power (10 W, max. 500 V, max. 100 mA) for 60 min. Then, gels were incubated in a freshly prepared 1:10000 ethidium bromide dilution for 15 min and then destained in dH₂O for 15 min. For visualization a Gel Doc EZ Imager (Bio Rad) was used.

### Oligonucleotides

Alkyne oligo1:

5′-TAA TGA TAC GGC GAC CAC CGA GAT CTA CAC TCT TTC
CCT ACA CGA CGC TCT TCC GAT CT-3′
T = 5′-alkyne dT

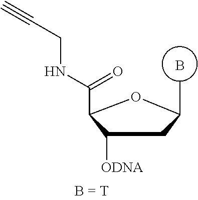

Azide oligo1:

5′-N₃-TGG AGT TCG TGA CCG CCG CCG GGA TCA CTC TCG
GCA TGG ACG AGC TGT ACA AGT AAA GC -3′

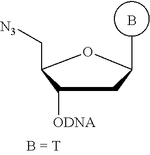

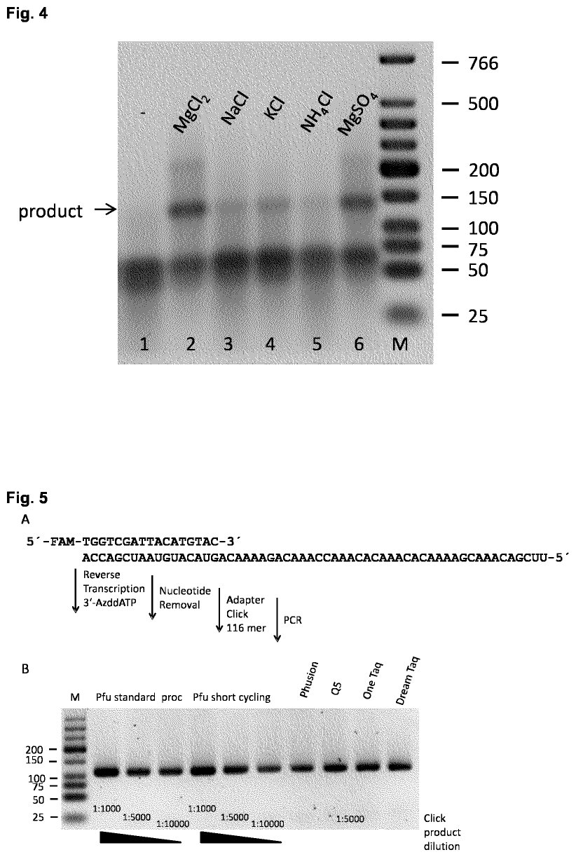

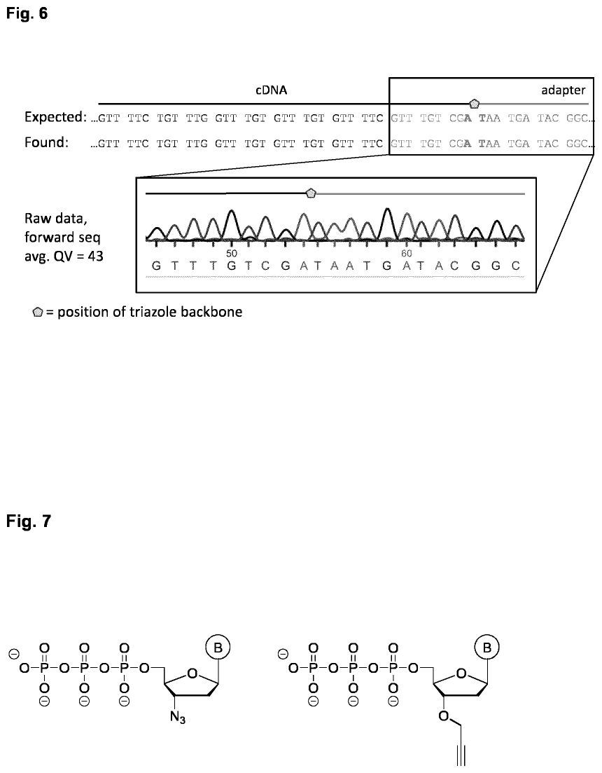

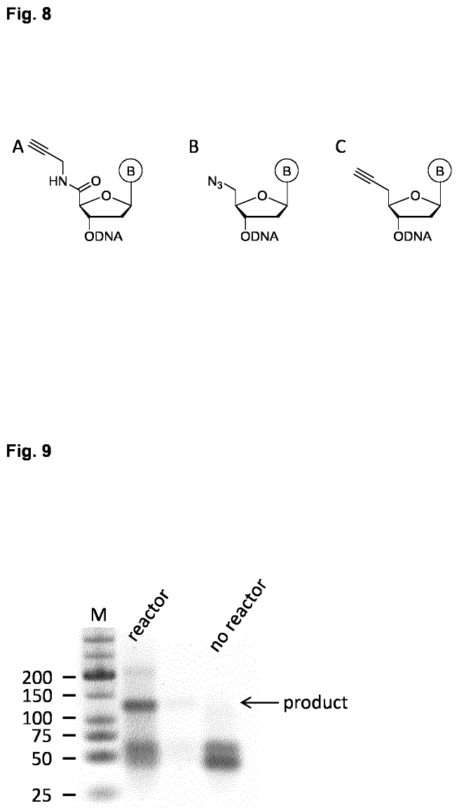

Due to the non-ideal click conditions used in this experiment (too much THPTA, not enough copper source), the influence of cation addition of (earth) alkaline metal addition becomes apparent. FIG. 4 shows the 2.5% gel of the oligonucleotide click reaction and the influence of different cations on the click efficiency and product yield. In the absence of an additional cation (slot 1) a yield of less than 5% of the click product was observed under the conditions described above. Through addition of Mg²⁺ ions (8 mM) the yield is improved to about 30%. As a comparison, a concentration of 16 mM of monovalent cations was also analyzed, however only a slight improvement of the yield was observed (slots 3-5, yields of 5 to 10%).

### EXAMPLE 2

### PCR Amplification of the Click Product

The feasibility of the ClickAdapt protocol is exemplified for a model RNA sequence. The RNA was hybridized to primer1 and then reverse transcribed in the presence of 200 μM dTTP, dGTP, dCTP and 3′-azido-ddATP using MuLV reverse transcriptase. Nucleotides and enzyme were removed by purification of the cDNA using the nucleotide removal kit (Qiagen) according to manufacturers' instructions.

Alkyne oligo1 was clicked to the purified cDNA in a 200 μL reaction vial with a single reactor pellet (600-800 μm, containing elemental copper) in a total 12.5 μL reaction mix and incubated at 45° C. for 60 min.

The reaction mix consisted of 800 μM THPTA, 20 mM MgCl₂, 5% (v/v) DMSO, 7 μM of alkyne oligol and about 4 μM purified cDNA. dH₂O was used to adjust the volume to a final 12.5 μL if necessary.

After the incubation the sample was briefly spinned down and the supernatant was transferred to a new vial to stop the reaction. The crude click reaction was diluted 1:1000, 1:5000 and 1:10000 (max. 4 nM, 0.8 nM and 0.4 nM) for PCR amplification without further purification.

In a 200 μL reaction vial, PCR amplifications were prepared in a total volume of 20 μL. Click reaction dilutions were combined with 200 μM dNTPs, 10 pmol of primer2 and primer3 and one unit polymerase. For the various polymerases, Pfu, Phusion, Q5, One Taq and Dream Taq buffers were used according to manufacturers' recommendations. The samples were subjected to a thermal cycling program in a thermocycler (BioRad).

As a standard cycling condition following conditions were used:

| step | temperature | duration | | | |
|---|---|---|---|---|---|
| 1 | 95° C. | 2 | min | | |
| 2 | 95° C. | 15 | s | | |
| 3 | 51° C. | 20 | s | {close oversize bracket} | 25 x |
| 4 | 72° C. | 30 | s | | |

| | | | |
|---|---|---|---|
| 5 | 72° C. | 2 | min |

For the Pfu polymerase different template dilutions and an alternative cycling condition were studied:

| step | temperature | duration | | | |
|---|---|---|---|---|---|
| 1 | 95° C. | 2 | min | | |
| 2 | 95° C. | 15 | s | | |
| 3 | 52° C. | 5 | s | {close oversize bracket} | 25 x |
| 4 | 72° C. | 20 | s | | |

| | | | |
|---|---|---|---|
| 5 | 72° C. | 2 | min |

After the incubation the sample was briefly spinned down and an aliquot was analyzed on 3% agarose gels (10×15 cm) prepared in TAE buffer (20 mM TRIS, 10 mM acetic acid, 0.5 mM EDTA).

Samples were prepared with 20% purple loading dye (NEB), and low molecular weight DNA ladder (25-766 bp, NEB, N3233) was prepared accordingly; usually 0.5 μL marker were used in 5 μL loading volume. Gels were run in TAE buffer applying constant power (10 W, max. 500 V, max. 100 mA) for 60 min. Then, gels were incubated in a freshly prepared 1:10000 ethidium bromide dilution for 15 min and then destained in dH₂O for 15 min. For visualization a Gel Doc EZ Imager (Bio Rad) was used.

FIG. 5A illustrates the ClickAdapt workflow (described above) which was done for the shown model RNA. The workflow involves reverse transcription of the RNA into cDNA. By replacing natural dATP by 3′-AzddATP the cDNA is terminated with a 3′-azide. After removal of excess nucleotides by purification of the cDNA, a 5′-alkyne adapter is click ligated to the 3′-azide and the crude reaction mix is used as template for PCR.

FIG. 5B is an ethidium bromide stained agarose gel of PCR samples treated according to the ClickAdapt workflow for the model RNA in 5A. Since the triazole-containing template is amplified by various polymerases under different conditions, this illustrates the biocompatibility of the unnatural backbone mimic.

### Oligonucleotides

Alkyne oligo1 (see example 1)

Model RNA:
5′-UUC GAC AAA CGA AAA CAC AAA CAC AAA CCA AAC AGA
AAA CAG UAC AUG UAA UCG ACC A-3′
Primer1 (for reverse transcription)
5′-FAM-TGG TCG ATT ACA TGT AC-3′; FAM fluorescein
Primer2
5′-TGG TCG ATT ACA TGT ACT GTT TT-3′
Primer3
5′-AGA TCG GAA GAG CGT CG-3′

Resulting cDNA after reverse transcription:

5′-FAM-TGG TCG ATT ACA TGT ACT GTT TTC TGT TTG GTT
TGT GTT TGT GTT TTC GTT TGT CGA-N₃

Resulting click product:

5′-FAM-TGG TCG ATT ACA TGT ACT GTT TTC TGT TTG
GTT TGT GTT TGT GTT TTC GTT TGT CGA TAA TGA TAC
GGC GAC CAC CGA GAT CTA CAC TCT TTC CCT ACA CGA
CGC TCT TCC GAT CT-3′
AT = A and T joined via backbone mimic B

Resulting PCR product:

5′-TGG TCG ATT ACA TGT ACT GTT TTC TGT TTG GTT
TGT GTT TGT GTT TTC GTT TGT CGA TAA TGA TAC GGC
GAC CAC CGA GAT CTA CAC TCT TTC CCT ACA CGA CGC
TCT TCC GAT CT-3′

### EXAMPLE 3

### Sequence Determination of the Amplification Product

The amplification product was analyzed by a process which determines the sequence of nucleobases in a nucleic acid. Adapter sequences that have been included allow for a hybridization of a complementary oligonucleotide, for immobilization, sequence determination or further amplification.

FIG. 6 shows the results of Sanger sequencing of one of the amplification products of example 2. It was determined that using the Phusion DNA polymerase, no mutations could be observed at or close by the position of the triazole backbone modification. The result indicates that the click reaction product can be successfully included in a PCR reaction to provide high amounts of PCR products in high efficiency and accuracy.

### EXAMPLE 4

### Low Concentration Click Ligation in the Presence and Absence of Reactor (Copper Source)

In a 200 μL reaction vial two reactor pellets (600-800 μm, containing elemental copper; sample 1) or no reactor pellets (sample 2) were combined with 12.5 μL reaction mix and incubated at 45° C. for 60 min.

The reaction mix consisted of 800 μM THPTA, 20 mM MgCl₂, 7 μM of alkyne oligol and 7 μM of azide oligol in dH₂O.

After the incubation the sample was briefly spinned down and the supernatant was transferred to a new vial to stop the reaction. Samples were analyzed on 3% agarose gels (10×15 cm) prepared in TAE buffer (20 mM TRIS, 10 mM acetic acid, 0.5 mM EDTA).

Samples were prepared with 20% purple loading dye (NEB), and low molecular weight DNA ladder (25-766 bp, NEB, N3233) was prepared accordingly; usually 0.5 μL marker were used in 5 μL loading volume. Gels were run in TAE buffer applying constant power (10 W, max. 500 V, max. 100 mA) for 60 min. Then, gels were incubated in a freshly prepared 1:10000 ethidium bromide dilution for 15 min and then destained in dH₂O for 15 min. For visualization a Gel Doc EZ Imager (Bio Rad) was used.

### Oligonucleotides

Alkyne oligo1:

5′-TAA TGA TAC GGC GAC CAC CGA GAT CTA CAC TCT
TTC CCT ACA CGA CGC TCT TCC GAT CT-3′
T = 5'-alkyne dT

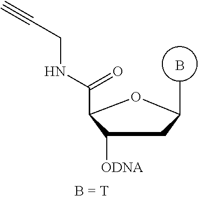

Azide oligo1:

5′-N₃-TGG AGT TCG TGA CCG CCG CCG GGA TCA CTC TCG
GCA TGG ACG AGC TGT ACA AGT AAA GC-3′

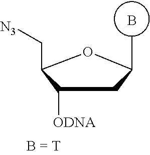

The results of this example are shown in FIG. 9. A yield of 36% was obtained after 60 min using the click condition of this example when the reactor was present. When the reactor was omitted, no product was observed.

### EXAMPLE 5

### RNA Library Preparation Protocol Using IVT mRNA

Here we describe detailed experimental conditions of library preparation protocols which have been obtained during protocol development using purified in vitro transcribed (IVT) mRNA coding for the eGFP gene.

### Reverse Transcription

In 200 μL RNase free tubes 250 ng IVT mRNA was combined with 100 pmol partly randomized primer, 1× reaction buffer, 10 mM DTT (dithiothreitol), 500 μM dNTP, 0-100 μM AzddNTP and 200 units reverse transcriptase.

A) Primer Hybridization Pipetting Scheme:

| | Setup | | | | | | | | | |
|---|---|---|---|---|---|---|---|---|---|---|
| Component | 1 | | 2 | | 3 | | 4 | | 5 | |
| H₂O | 7.35 | μL | 6.95 | μL | 6.55 | μL | 5.35 | μL | 6.35 | μL |
| Illumina_N6 | 1 | μL | 1 | μL | 1 | μL | 1 | μL | 1 | μL |
| primer | | | | | | | | | | |
| (100 μM) | | | | | | | | | | |
| eGFP | 0.65 | μL | 0.65 | μL | 0.65 | μL | 0.65 | μL | 0.65 | μL |
| mRNA | | | | | | | | | | |
| (382 ng/μL) | | | | | | | | | | |
| dNTP | 1 | μL | 1 | μL | 1 | μL | 1 | μL | 1 | μL |
| (10 mM) | | | | | | | | | | |

| | | | | | | |
|---|---|---|---|---|---|---|
| AzddNTP | — | — | — | — | 1.0 | μL |
| (2 mM) | | | | | | |

| | | | | | | | | |
|---|---|---|---|---|---|---|---|---|
| AzddNTP | — | 0.4 | μL | 0.8 | μL | 2.0 | μL | — |
| (500 μM) | | | | | | | | |

The components were mixed by gently pipetting and then heated in thermocycler to 65° C. for 3 min and then cooled to 4° C. For addition of the remaining components a master mix (for 6 setups) was prepared:

| Component | Volume |
|---|---|
| H₂O | 18 μL |
| Reverse transcription buffer¹ (5x) | 24 μL |
| DTT¹ (100 mM = 10x) | 12 μL |
| Protoscript II reverse transcriptase¹ (200 U/μL) | 6 μL |

¹= from NEB (product number M0368L)

To each hybridized setup (1-5) were added 10 μL master mix at room temperature (23° C.) and after mixing by pipetting the reverse transcriptions were incubated in a thermocycler at 25° C. for 10 min, 42° C. (optimum temperature for protoscript II rt enzyme) for 50 min and 65° C. (denaturation) for 20 min. After cooling to 4° C., 5 μL NaOH_{(aq)} (1 M) was added to each setup and then incubated at 95° C. for 15 min, then 4° C. The mixtures were neutralized by addition of 5 μL HCl(_{aq}) (1 M) and then purified using the Qiagen PCR purification kit (addition of 150 μL PB buffer, final elution step using 30 μL H₂O) according to manufacturer's recommendations.

### NanoDrop Measurement

| | | β | | total | estimated |
|---|---|---|---|---|---|
| Setup | A₂₆₀ | [ng/μL] | comment | volume | cDNA amount |
| 1 | 0.058 | 21.1 | intense DNA spectrum | 29 μL | 610 ng |
| 2 | 0.054 | 19.6 | intense DNA spectrum | 28 μL | 540 ng |
| 3 | 0.044 | 15.9 | typical DNA spectrum | 29 μL | 460 ng |
| 4 | 0.033 | 11.1 | typical DNA spectrum | 29 μL | 320 ng |
| 5 | 0.037 | 13.3 | typical DNA spectrum | 28 μL | 370 ng |

An increased AzddNTP concentration during reverse transcription decreases cDNA yield. We assume that an increased AzddNTP amount decreases the fragment size and fragments<100 mer are removed during cDNA purification.

### Click Ligation

In a 200 μL reaction tube two reactor pellets were combined with 1.25 μL of activator (200 mM MgCl₂, 8 mM THPTA in 50% (v/v) aqueous DMSO) (10×), 1 μL of alkyne adapter oligo (100 μM) and 10.25 μL of purified cDNA for each setup (1-5). The mixture was incubated in a thermomixer at 45° C., 600 rpm for 60 min. Then each sample was briefly spinned down and the supernatant was transferred to a fresh vial.

### PCR

In a 200 μL reaction tube 20 μL PCRs were prepared by combining 0.5 μL click reaction, 10 pmol primer, 0.5 units Phusion DNA polymerase in Phusion buffer and 200 μM dNTPs.

A master mix was prepared for 6 setups:

| Component | Volume | |
|---|---|---|
| H₂O | 75.6 | μL |
| Phusion HF buffer³ (5x) | 24 | μL |
| P17-006_forw2 primer (10 μM) | 6 | μL |
| P17-006_rev3 primer (10 μM) | 6 | μL |
| dNTPs (10 mM) | 2.4 | μL |
| Phusion Polymerase³ (1 U/μL) | 3 | μL |

³= from THERMO FISHER SCIENTIFIC, product number F530L

The components were mixed by gently pipetting and 19.5 μL master mix was added to 0.5 μL of finished click reaction (without pellet!) and mixed. The resulting mixtures were incubated in a thermocycler applying following temperature program:

| T [° C.] | t [s] | | |
|---|---|---|---|
| 98 | 30 | | |
| 98 | 10 | | |
| 52 | 10 | {close oversize bracket} | 30x |
| 72 | 40 | | |

| | |
|---|---|
| 72 | 120 |

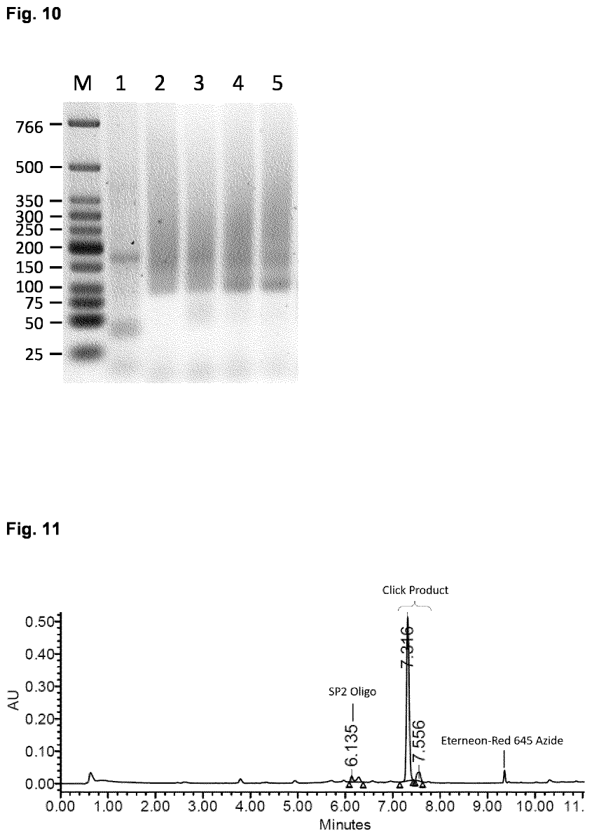

5 μL of each PCR sample were analyzed by agarose gel electrophoresis. Samples were loaded starting with dNTPs only (setup 1, lane 1) during initial reverse transcription, which provided a weak single band at 180 bp and the primers (around 35 bp). PCR samples from cDNA, which were generated from AzddNTP incorporation during reverse transcription resulted in a smear (lane 2-5, setup 5-2) from 100 bp (size cut-off of purification method) to 700 bp (lane 5). Fragment size distribution seemed to increase from lane 3-5, as the AzddNTP amount was decreased during reverse transcription from 50 μM to 10 μM (lane 3 to 5). The ethidium bromide stained agarose gel is shown in FIG. 10.

### Oligonucleotides

| Oligonucleotide | Sequence (5′ to 3′) |
|---|---|
| Illumina_N6 primer | GTGACTGGAGTTCAGACGTGTGCTCTTCC |
| (100 μM) | GATCTNNNNNN |
| alkyne adapter oligo | TAATGATACGGCGACCACCGAGATCTACA |
| | CTCTTTCCCTACACGACGCTCTTCCGATC |
| | T |
| P17-006_forw2 primer | GTGACTGGAGTTCAGACGTG |
| (10 μM) | |
| P17-006_rev3 primer | GTCGTGTAGGGAAAGAGTGTA |
| (10 μM) | |

T = 5′-alkyne dT

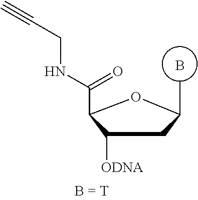

### EXAMPLE 6

### Oligo-Dye Click Reaction Using Metal Cations

In a 1500 μL reaction vial four reactor pellets were combined with 27.9 μL reaction mix and were incubated at 45° C. for 60 min.

The reaction mix consisted of 17.9 mM MgSO₄, 71.7 μM SP2-Oligo, 1.8 mM THPTA, 144 μM Eterneon-Red 645 Azide in 2.9 μL DMSO and 25 μL H₂O.

After the incubation the sample was briefly spinned down and the supernatant was transferred to a new vial. An aliquot of the sample was diluted in dH₂O and then analyzed by analytical HPLC. Analytical RP-HPLC was performed on an analytical HPLC WATERS Alliance (e2695 Separation Module, 2998 Photodiode Array Detector) equipped with the column XBridge™ OST C18 (2.5 μm, 4.6×50 mm) from WATERS. Using a flow of 1.5 mL/min and a column temperature of 40° C. following gradient was used for separation of click reactions: 0-30% B in 8 min, 85% B after 10 min, 100% B in 11 min. Buffer A: 0.1 M triethylammonium acetate in water, pH=7, buffer B: 0.1 M triethylammonium acetate in 80% (v/v) acetonitrile, pH=7. A detection range from 220-680 nm was used for the runs.

An alkyne-modified oligonucleotide was reacted with Eterneon Red 645 Azide using MgSO₄ as an additive in addition to copper from the reactor pellet and the ligand. In the presence of MgSO₄ a click product conversion yield of 85% was obtained for the crude reaction after 60 min incubation (FIG. 11).

### EXAMPLE 7

### HPL Chromatogram of an Oligo-Oligo Click

In a 200 μL reaction vial one reactor pellet (600-800 μm, containing elemental copper) was combined with 6.7 μL reaction mix and incubated at 45° C. for 60 min. The reaction mix consisted of 800 μM THPTA, 20 mM MgCl₂, 455 μM alkyne oligo biotin and 450 μM azide oligo biotin in dH₂O with 5% DMSO (v/v).

After the incubation the sample was briefly spinned down and the supernatant was transferred to a new vial. An aliquot of the sample was diluted in dH₂O and then analyzed by analytical HPLC. Analytical RP-HPLC was performed on an analytical HPLC WATERS Alliance (e2695 Separation Module, 2998 Photodiode Array Detector) equipped with the column XBridge™ OST C18 (2.5 μm, 4.6×50 mm) from WATERS. Using a flow of 1.5 mL/min and a column temperature of 40° C. following gradient was used for separation of click reactions: 0-30% B in 8 min, 85% B after 10 min, 100% B in 11 min. Buffer A: 0.1 M triethylammonium acetate in water, pH=7, buffer B: 0.1 M triethylammonium acetate in 80% (v/v) acetonitrile, pH=7. A detection range from 220-680 nm was used for the runs.

A reaction between a singly alkyne and singly azide modified oligonucleotide was studied by analytical HPLC, since the retention time of the alkyne-modified oligonucleotide (Rₜ=5.62 min) was very different from the azide-modified oligonucleotide (Rₜ=7.64 min). After 60 min at 45° C., almost the complete amount of azide-modified oligonucleotide was consumed. Two major new peaks were observed at 6.10 and 6.38 min, which had the mass of the desired click product in subsequent ESI-MS. Due to a slight excess of the alkyne-modified oligonucleotide, a distinct peak of the alkyne oligo was observed at the end of the reaction (5.618 min).

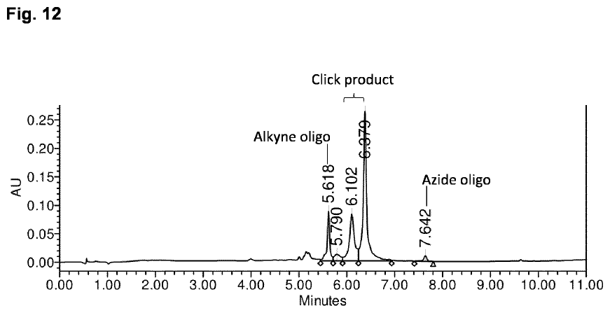

Peak table of the integrated peaks shown in FIG. 12.

| Entry | Rₜ [min] | Area | Height | % Area |
|---|---|---|---|---|
| 1 | 5.618 | 337824 | 86604 | 13.25 |
| 2 | 5.790 | 102820 | 11520 | 4.03 |
| 3 | 6.102 | 627408 | 81837 | 24.60 |
| 4 | 6.379 | 1434201 | 262451 | 56.24 |
| 5 | 7.642 | 47989 | 9893 | 1.88 |

### Oligonucleotides

| Oligo- | | |
|---|---|---|
| nucleotide | Sequence (5′ to 3′) | Modification |
| Alkyne oligo | Biotin-TEG-GTT CTA | X = C8-alkyne |
| biotin | GAA CCC TAA GAA AAA | dU |
| | TCT CXA CCA | |
| Azide oligo | Biotin-TEG-GTT CTA | Y = PEG12 |
| biotin | GAA CCC TAA GAA AAA | azide amino |
| | TCT CYA CCA | dT |

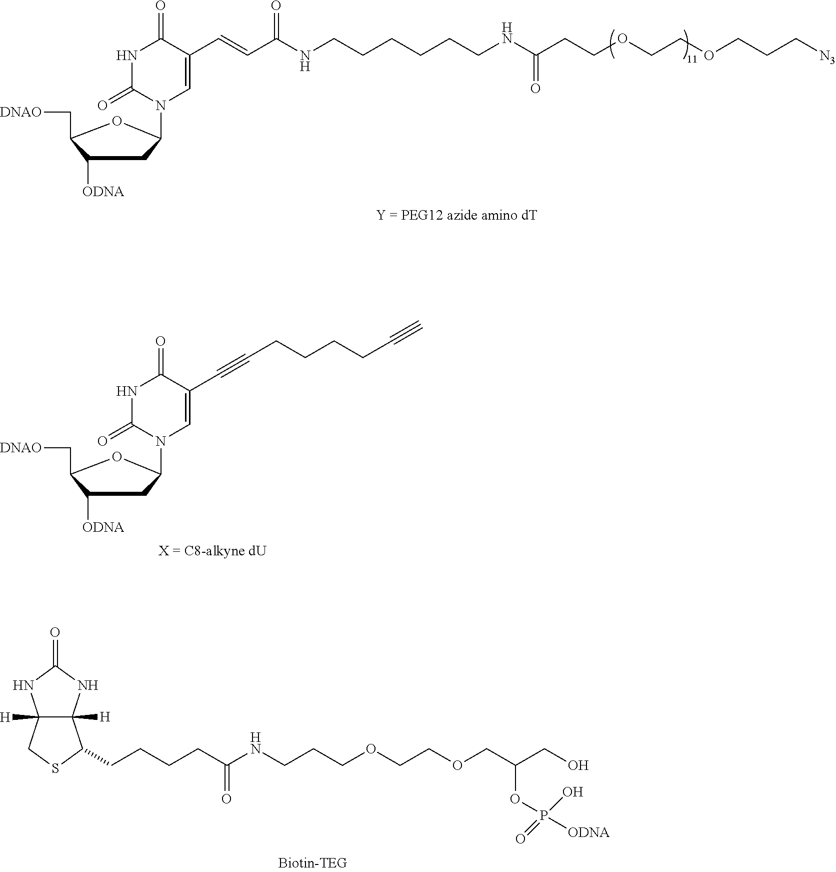

1. A method for coupling a first molecule to a second molecule in a click ligation reaction, wherein the first molecule comprises a first click functional group which is an alkyne group, and the second molecule comprises a second click functional group, which is an azide group, the method comprising contacting the first and second molecules in a reaction mixture in the presence of a catalyst, characterized in that the click reaction is performed in the presence of additional metal cations in the reaction mixture.

2. A method according to claim 1, wherein as additional metal cations alkaline or earth alkaline metal cations, preferably Li⁺, K⁺, Mg²⁺, or Zn²⁺ are present in the click reaction mixture.

3. A method according to claim 1, wherein the metal cations are contained in the click reaction mixture in an amount of 1 to 200 mmol/l, preferably 5 to 25 mmol/l and most preferably 10 to 20 mmol/l.

4. A method according to anyone of claim 1, wherein the click reaction mixture comprises an organic solvent, preferably DMSO, and/or a Cu-stabilizing ligand, preferably selected from at least one of Tris(3-hydroxypropyltriazolylmethyl)amine (THPTA), 2-(4-((bis((1-(tert-butyl)-1H-1,2,3-triazol-4-yl)methyl)amino)methyl)-1H-1,2,3-triazol-1-yl)acetic acid (BTTAA), Tris((1-benzyl-4-triazolyl)methyl)amine (TBTA), 2-(4-((bis((1-(tert-butyl)-1H-1,2,3,-triazol-4-yl)methyl)amino)methyl)-1H-1,2,3-triazolyl-1-yl) ethyl sulfate (BTTES) or analogs thereof, especially other trident polytriazoles.

5. A method according to claim 4, wherein the organic solvent is contained in the click reaction mixture in an amount of 2 to 10% (v/v), preferably 4 to 6% (v/v) and/or the Cu(I)-stabilizing ligand is contained in the click reaction mixture in an amount of 10 to 4000 μmol/l, preferably 500 to 1000 μmol/l.

6. A method according to anyone of claim 1, wherein the catalyst is a Cu catalyst, preferably a heterogeneous Cu catalyst.

7. A method according to anyone of claim 1, wherein at least one of the first and second molecules is a biomolecule, preferably selected from nucleosides, nucleotides, nucleic acids, amino acids, peptides, saccharides and lipids and wherein especially preferably both the first and the second molecules are oligonucleotides.

8. A method according to anyone of claim 1, wherein at least one of the first and second molecules carries a detectable label.

9. An activator composition for use in a click ligation reaction wherein a first molecule comprising a first click functional group, which is an alkyne group, and a second molecule comprising a second click functional group, which is an azide group, are coupled in the presence of a catalyst, preferably a heterogeneous Cu catalyst, said activator mixture comprising additional metal cations and further an organic solvent and/or a Cu-stabilizing ligand.

10. The activator composition according to claim 9, wherein the divalent metal cations are earth alkaline metal cations, preferably Mg²⁺, and/or the Cu-stabilizing ligand is selected from at least one of Tris(3-hydroxypropyltriazolylmethyl)amine (THPTA), 2-(4-((bis((1-(tert-butyl)-1H-1,2,3-triazol-4-yl)methyl)amino)methyl)-1H-1,2,3-triazol-1-yl)acetic acid (BTTAA), Tris((1-benzyl-4-triazolyl)methyl)amine (TBTA), 2-(4-((bis((1-(tert-butyl)-1H-1,2,3,-triazol-4-yl)methyl)amino)methyl)-1H-1,2,3-triazolyl-1-yl) ethyl sulfate (BTTES) or analogs thereof, especially other trident polytriazoles, and/or the organic solvent is DMSO.

11. The activator composition according to claim 9, comprising the metal cation in an amount of 1 to 200 mmol/l, preferably 5 to 25 mmol/l, and/or the organic solvent in an amount of 2 to 10% (v/v), preferably 4 to 6% (v/v), and/or the Cu-stabilizing ligand in an amount of 10 to 4000 μmol/l, preferably 500 to 1000 μmol/l.

12. Click ligation reagent kit, comprising as one component a heterogenous Cu catalyst and as a second component an activator composition according to any one of claim 9.

13. Click ligation reagent kit according to claim 12, comprising one or more further components of a click reaction, preferably selected from the group consisting of a first molecule comprising a first Click functional group, which is an alkyne group, a second molecule comprising a second Click functional group, which is an azide group, buffers, solvents, enzymes, modified and/or non-modified nucleotides, (index) primer(s) and/or adapters optionally including a double-stranded loop at the 5′ end, and optionally chromatographic materials.

14. A device having at least one reaction chamber comprising a heterogeneous Cu catalyst for a click ligation reaction for coupling a first molecule to a second molecule, wherein the first molecule comprises a first click functional group which is an alkyne group, and the second molecule comprises a second click functional group, which is an azide group, and optionally a further solid carrier material, wherein at least in one reaction chamber of the device metal cations or an activator composition according to claim 9 are present.

15. Use of the method according to claim 1, further including subsequent reactions selected from RNA or DNA amplification, RNA or DNA labelling methods and RNA or DNA sequencing methods.


SEQUENCE LISTING (18 sequences)

SEQ ID NO: 1 - DNA, 59 bp, Artificial Sequence
  Alkyne oligo1
  modified_base (1)..(1): 5-prime-alkyne dT
taatgatacggcgaccaccgagatctacactctttccctacacgacgctcttccgatct

SEQ ID NO: 2 - DNA, 59 bp, Artificial Sequence
  Azide oligo1
  modified_base (1)..(1): 5-prime-azide dT
tggagttcgtgaccgccgccgggatcactctcggcatggacgagctgtacaagtaaagc

SEQ ID NO: 3 - RNA, 58 nt, Artificial Sequence
  artificial model RNA sequence to show feasibility of ClickAdapt protocol according to invention
uucgacaaacgaaaacacaaacacaaaccaaacagaaaacaguacauguaaucgacca

SEQ ID NO: 4 - DNA, 17 bp, Artificial Sequence
  reverse transcription Primer1
  modified_base (1)..(1): 5-prime-FAM-dT (FAM=Fluorescein)
tggtcgattacatgtac

SEQ ID NO: 5 - DNA, 23 bp, Artificial Sequence
  reverse trascription Primer2
tggtcgattacatgtactgtttt

SEQ ID NO: 6 - DNA, 17 bp, Artificial Sequence
  reverse transcription Primer3
agatcggaagagcgtcg

SEQ ID NO: 7 - DNA, 57 bp, Artificial Sequence
  cDNA after reverse transcription
  modified_base (1)..(1): 5-prime-FAM-dT (FAM=Fluorescein)
  modified_base (57)..(57): 3-prime-azide dA
tggtcgattacatgtactgttttctgtttggtttgtgtttgtgttttcgtttgtcga

SEQ ID NO: 8 - DNA, 116 bp, Artificial Sequence
  click product after nucleotide removal and adapter click on SEQ ID NO.7
  modified_base (1)..(1): 5-prime-FAM dT (FAM=Fluorescein)
  misc_feature (62)..(63): A and T joined via backbone mimic B
tggtcgattacatgtactgttttctgtttggtttgtgtttgtgttttcgtttgtcgataatgatacggcgaccaccgagatctacactctttccctacacgacgctcttccgatct

SEQ ID NO: 9 - DNA, 116 bp, Artificial Sequence; identical to SEQ ID NO: 8
  PCR product

SEQ ID NO: 10 - DNA, 51 bp, Artificial Sequence
  Fig.6 expected sequence after amplification (reverse transcription, click reaction, PCR)
gttttctgtttggtttgtgtttgtgttttcgtttgtcgataatgatacggc

SEQ ID NO: 11 - DNA, 51 bp, Artificial Sequence; identical to SEQ ID NO: 10
  Fig.6 found sequence after amplification (reverse transcription, click reaction, PCR)

SEQ ID NO: 12 - DNA, 21 bp, Artificial Sequence
  Fig.6 forward seq avg. QV=43, determined by Sanger sequencing, partial sequence of SEQ ID NO.11
gtttgtcgataatgatacggc

SEQ ID NO: 13 - DNA, 40 bp, Artificial Sequence
  Illumina N6 primer
  misc_feature (35)..(40): n is a, c, g, or t
gtgactggagttcagacgtgtgctcttccgatctnnnnnn

SEQ ID NO: 14 - DNA, 59 bp, Artificial Sequence; identical to SEQ ID NO: 1
  Alkyne adapter oligo
  modified_base (1)..(1): 5-prime-alkyne dT

SEQ ID NO: 15 - DNA, 20 bp, Artificial Sequence
  P17-006 forw2 primer
gtgactggagttcagacgtg

SEQ ID NO: 16 - DNA, 21 bp, Artificial Sequence
  P17-006 rev3 primer
gtcgtgtagggaaagagtgta

SEQ ID NO: 17 - DNA, 30 bp, Artificial Sequence
  Alkyne oligo biotin
  modified_base (1)..(1): 5-prime-Biotin-TEG-dG
  modified_base (26)..(26): C8-alkyne dU
gttctagaaggctaagaaaaatctcuacca

SEQ ID NO: 18 - DNA, 30 bp, Artificial Sequence
  Azide oligo biotin
  modified_base (1)..(1): 5-prime-Biotin-TEG-dG
  modified_base (26)..(26): PEG12 azide amino dT
gttctagaaggctaagaaaaatctctacca